(19) United States
(12) Patent Application Publication
(10) Pub. No.: US 2025/0032855 A1
(43) Pub. Date: Jan. 30, 2025

(54) Virtual Personal Trainer

(71) Applicant: Kenneth Eade, Monaco (MC)

(72) Inventor: Kenneth Eade, Monaco (MC)

(21) Appl. No.: 18/225,178

(22) Filed: Jul. 24, 2023

(51) Int. Cl. A63B 24/00; A63B 71/06

(52) U.S. Cl. CPC A63B 24/0075; A63B 24/0003; A63B 24/0062; A63B 71/0622; A63B 2071/0638; A63B 2071/0666; A63B 2225/20; A63B 2230/06

(57) ABSTRACT

The present invention discloses a system and method for interactive personal training using virtual reality (VR) and artificial intelligence (AI). The system comprises a VR headset that offer users a realistic training environment, a computer that tracks the user's movements, and a database of actual videos featuring personal trainers demonstrating exercises. Users can watch instructional videos and replicate the exercises in the VR environment. The AI algorithms analyze the user's movements and provide personalized feedback on form and technique, allowing users to benefit from professional guidance remotely.

### TECHNICAL FIELD

The present invention relates to the field of interactive personal training. Specifically, it pertains to a system and method for providing interactive personal training using virtual reality (VR) and artificial intelligence (AI) technologies.

### BACKGROUND

Virtual reality and artificial intelligence have seen significant advancements in recent years and have been utilized in various industries, including the fitness and wellness sectors. VR technology allows users to immerse themselves in realistic and interactive environments, while AI algorithms can provide personalized feedback and guidance based on user inputs and movements.

Existing personal training systems using VR and AI focus on creating realistic training environments and providing personalized feedback. However, there is a need for a system that combines the benefits of VR, AI, and actual personal trainers to offer users the opportunity to watch and learn from professionals.

### SUMMARY OF INVENTION

The present invention introduces a groundbreaking system and method for interactive personal training utilizing VR and AI technologies. By combining VR headsets, a computer for motion tracking, and a database of personal trainers' instructional videos, users can immerse themselves in a realistic training environment, learn from professional trainers, and receive personalized feedback on their form and technique. This system and method offer convenience, personalization, and engagement, revolutionizing the way individuals engage in personal fitness and training.

The system includes a VR headset that enables users to experience a realistic training environment and a computer that tracks the user's movements. The system further comprises a database containing actual videos of personal trainers demonstrating exercises.

The user can watch a video of a personal trainer demonstrating an exercise and then replicate the exercise in the VR environment. The AI algorithms analyze the user's movements and provide personalized feedback on their form and technique. This enables users to learn from professional trainers and receive personalized guidance, even in the absence of an in-person trainer.

1. A system for providing interactive personal training using Virtual Reality (VR) and Artificial Intelligence (AI), comprising:
a. A VR headset that provides users with a realistic training environment;
b. A computer that tracks the users movements during training;
c. A database containing actual videos of personal trainers demonstrating exercises; and
d. A software program that:
i. Displays the VR training environment to the user through the VR headset;
ii. Tracks the user's movements during exercise performance;
iii. Provides personalized feedback to the user based on their movements; and
iv. Enables users to watch instructional videos of personal trainers and replicate the exercises in the VR environment.

2. The system of claim 1, wherein the software program further includes a feature for adjusting the difficulty of the training based on the user's progress.

3. The system of claim 1, wherein the software program further includes a feature for tracking the user's heart rate and providing feedback based on the users heart rate.

4. The system of claim 1, wherein the software program is configured to be downloaded to the computer from a remote server.

5. A method for providing interactive personal training using VR and AL, comprising the steps of:
a. Displaying a realistic training environment to a user through a VR headset;
b. Tracking the user's movements during exercise performance using a computer;
c. Providing personalized feedback to the user based on their movements;
d. Accessing a database containing actual videos of personal trainers demonstrating exercises;
e. Allowing the user to watch instructional videos of personal trainers;
f. Enabling the user to replicate the exercises demonstrated in the videos within the VR environment.

6. The method of claim 5, further comprising the step of adjusting the difficulty of the training based on the user's progress.

7. The method of claim 5, further comprising the step of tracking the user's heart rate and providing feedback based on the user's heart rate.

8. The method of claim 5, wherein the software program is downloaded to the computer from a remote server.

9. The method of claim 5, further comprising the step of providing personalized feedback to the user based on an analysis of their performance in the VR training environment.

10. The method of claim 5, further comprising the step of recording and analyzing user data to improve the accuracy of personalized feedback and training recommendations.

11. The method of claim 5, further comprising the step of providing the user with progress reports and performance analytics based on their training sessions.
